(19) United States
(12) Patent Application Publication
(10) Pub. No.: US 2026/0365961 A1
(43) Pub. Date: Nov. 19, 2026

(54) MECHANICAL STRETCHING SYSTEM FOR MIMICKING PHYSIOLOGICAL ENVIRONMENT

(71) Applicant: IFOM - ISTITUTO FONDAZIONE DI ONCOLOGIA MOLECOLARE ETS, Milano (IT)

(72) Inventor: Qingsen LI, Milano (IT)

(73) Assignee: IFOM - ISTITUTO FONDAZIONE DI ONCOLOGIA MOLECOLARE ETS, Milano (IT)

(21) Appl. No.: 19/133,492

(22) PCT Filed: Dec. 1, 2023
(86) PCT No.: PCT/EP2023/083868

§ 371 Date: May 28, 2025

(30) Foreign Application Priority Data
Dec. 2, 2022 (EP) 22211187.4

(51) Int. Cl. C12M 1/42; C12M 1/00; C12M 1/12

(52) U.S. Cl. CPC C12M 35/04; C12M 23/26; C12M 25/02

(57) ABSTRACT

A cell stretching device is provided including a housing having walls defining a hollow region, first and second deformable membranes fixable to the walls of the housing, and an air chamber in an internal region of the housing is provided. The central portion of the walls include a vacuum port to evacuate air from the air chamber and to determine pressure variation inside the air chamber. In one configuration, the air pressure inside the air chamber causes the first deformable membrane to be separated from the second deformable membrane and in a second configuration, the air pressure inside the air chamber is reduced, deforming the deformable membranes and stretching them towards the central portion of the walls of the housing and such that the first deformable membrane is in contact with the second deformable membrane to form a stretching region.

### TECHNICAL FIELD

The invention relates to a cell stretching device and to a cell stretching plate comprising a plurality of said cell stretching devices. Also, the invention relates to a method for cell stretching using said device or said plate and to a process for retrofitting a cell stretching system using a vacuum unit.

### BACKGROUND ART

Cells in the body constantly experience mechanical stimuli in various organ movement, development and functions. These mechanical stimuli are often essential for several cell functions, such as migration, proliferation, differentiation. Cells sense such mechanical stimuli through a process called mechanotransduction. Abnormal mechanotransduction may lead to several pathologies, such as cancer, asthma, and heart disease. In mechanobiology studies, the advance of in vitro technologies for the application of physiologically mimicking mechanical force to cultured cells and tissue are vital to mimic the complex in vivo biological system. The growing interest in mechanobiology and technology gap have motivated the development of cell stretching devices in various research labs and companies.

In particular, U.S. Pat. No. 6,218,178 and U.S. Pat. No. 6,645,759 disclose both cell stretching systems that use a vacuum to stretch the membrane. However, these type of cell stretching systems require a loading post to support and maintain the membrane flat in the stretching region. This may lead to some disadvantages. First of all, the loading post underneath the membrane can limit the maximum stretching level and can make the system unsuitable for imaging. Also, the friction between the loading post and the membrane requires lubricant, which affects the stretching precision. In addition, the system cannot be miniaturized for high throughput screening due to loading post and friction.

Examples of the present disclosure seek to address or at least alleviate the above problems.

### DISCLOSURE OF THE INVENTION

According to a first aspect of the invention there is provided a cell stretching device comprising:
a housing having walls defining a hollow region;
a first deformable membrane and a second deformable membrane facing the first deformable membrane, both the first deformable membrane and the second deformable membrane being located in the housing and being fixable to the walls of the housing; and
an air chamber formed in an internal region of the housing and defined by the first deformable membrane, the second deformable membrane, and a central portion of the walls of the housing, said central portion of the walls comprising at least a vacuum port for evacuating air from the air chamber and to determine a pressure variation inside the air chamber,
wherein in a first configuration the air pressure inside the air chamber is such that the first deformable membrane is separated from the second deformable membrane at a predefined membrane distance, and in a second configuration the air pressure inside the air chamber is reduced such that the first deformable membrane and the second deformable membrane are deformed and stretched towards the central portion of the walls of the housing and such that the first deformable membrane is, at least partially, in contact with the second deformable membrane to form a stretching region.

In a second aspect of the invention there is provided a plate for cell stretching comprising:
a plurality of cell stretching devices according to the first aspect; and
at least a frame configured to position each of the plurality of cell stretching devices arranged in rows and columns.

In a third aspect of the invention there is provided a method for cell stretching using the cell stretching device according to the first aspect, or the plate according to the second aspect, the method comprising:
providing at least one cell stretching device or the plate for cell stretching;
seeding one of the first or the second deformable membrane with cells to be analyzed, thereby forming a deformable cell substrate;
incubating the cells for a first predetermined period of time; and
stretching the cell substrate by cyclic aspiration and release of air through the vacuum port of the device or plate for a second period of time.

In a fourth aspect of the invention there is provided a process for retrofitting a cell stretching system that uses a vacuum unit for generating a stretching on a deformable cell substrate, said cell stretching system comprising an apparatus supporting the deformable cell substrate and being configured to be connected to said vacuum unit, the process comprising replacing the apparatus with the cell stretching device according to the first aspect or the cell stretching plate according to the second aspect by connecting the vacuum unit to the vacuum port of the cell stretching device.

Other aspects and features are defined in the appended claims.

Examples of the disclosure may make it possible to carry out a stretching of cells in an in-vitro context using a device functioning without loading posts. Accordingly, the device is easy to use and at the same time precise in applying a stretching force to the cells. Also, the device is suitable for imaging due to an increased maximum stretching level and can be miniaturized for high throughput screening.

### BRIEF DESCRIPTION OF THE DRAWINGS

Examples of the disclosure will now be described by way of example only with reference to the accompanying drawings, in which like references refer to like parts, and in which:

FIG. 1 shows a schematic representation of the cell stretching device according to an example.

FIGS. 2A-B show a schematic perspective representation of the cell stretching device according to an example in exploded view (FIG. 2A) and according to another example (FIG. 2B).

FIGS. 3A-C show a schematic representation of the plate according to two examples.

FIG. 4A-B show a configuration characterization of the cell stretching device according to examples.

FIG. 5 shows a flow chart of a cell stretching method according to an example.

FIG. 6 shows a schematic representation of the steps of a retrofitting process according to an example.

FIG. 7 shows a schematic representation of the cell stretching device according to another example.

FIG. 8 shows a schematic representation of the cell stretching device according to a further example.

### DETAILED DESCRIPTION OF ILLUSTRATIVE EMBODIMENTS

A cell stretching device, a plate, a method for cell stretching as well as a retrofitting process are disclosed. In the following description, a number of specific details are presented in order to provide a thorough understanding of the examples of the disclosure. It will be apparent however to a person skilled in the art that these specific details need not be employed in order to practice the examples of the disclosure. Conversely, specific details known to the person skilled in the art are omitted for the purposes of clarity in presenting the examples.

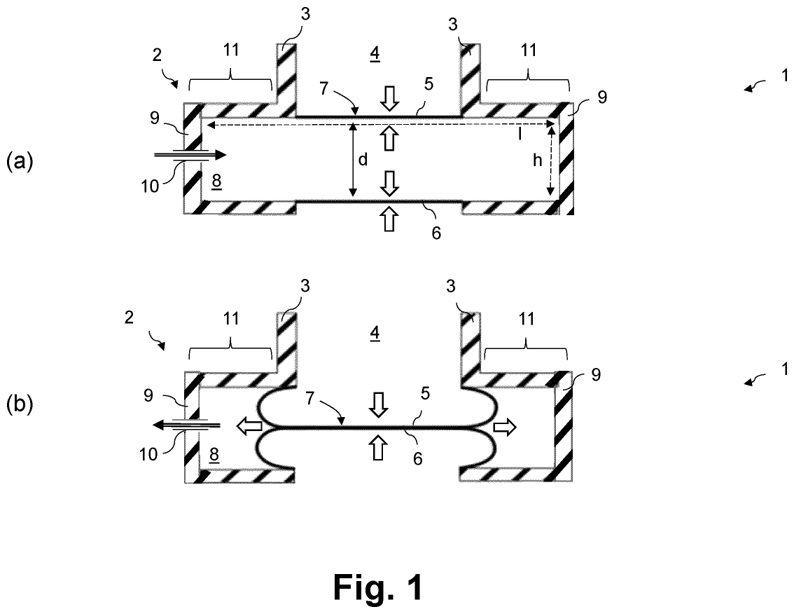

With reference to FIG. 1, a cell stretching device 1 is shown. The cell stretching device 1 comprises a housing 2 having basically a cylindrical shape defined by confining walls 3. In principle, the housing 2 can assume any other suitable shape providing that the housing 2 has a hollow structure and comprises a hollow region 4. The device 1 comprises two deformable membranes, a first membrane 5 and a second membrane 6 both fixed to the walls 3 of the housing 2. The first membrane 5 faces the second membrane 6 and defines, together with a central portion 9 of the walls 3 of the housing 2, an air chamber 8. In other words, the air chamber 8 is formed between the two membranes 5, 6 and the central portion 9 of the walls 3 of the housing 2 in an internal region of the housing 2. In order to evacuate air from the air chamber 8 and therefore to change, in particular reduce, the air pressure inside the chamber 8, the central portion 9 comprises at least a vacuum port 10. Accordingly, the air chamber 8 can be connected to a vacuum unit comprising a vacuum pump through the vacuum port 10. As the air chamber 10 is vacuumed, the membranes 5, 6 are deformed by atmospheric pressure and press/support each other to form a flat and stretched region.

Referring to FIG. 1, in a first configuration (a), the air pressure inside the air chamber 8 is such that the two membranes 5, 6 are not deformed. For example, air is not evacuated (or partially evacuated) from the air chamber 8, meaning that the air chamber 8 is at the atmospheric pressure or at a pressure value close to the atmospheric pressure such that the pressure exerted on the internal surfaces of the two membranes 5, 6 compensates (or almost compensate) the pressure exerted on the external surfaces of the two membranes 5, 6, as schematically shown by the vertical opposing arrows in the figure. The internal surface of the membranes 5, 6 indicates the side of the membranes 5, 6 facing the air chamber 8, whereas the external surface of the membranes 5, 6 indicates the side of the membranes 5, 6 opposite to the internal surface, i.e. outside the air chamber 8. The arrow shown in the vacuum port 10 pointing to the right in the figure means that in the first configuration air can be inserted into the air chamber 8 from outside in order to compensate the atmospheric pressure exerted on the external surfaces of the first and second membranes 5, 6, for example if the air was first evacuated from the chamber 8. In the first configuration (a), the first deformable membrane 5 is separated from the second deformable membrane 6 at a predefined membrane distance d. If the pressure inside the air chamber 8 equals the external pressure on the membranes 5, 6, e.g., the atmospheric pressure, the two membranes 5, 6 are flat and the membrane distance d basically corresponds to the height h of the air chamber 8.

By reducing the air pressure inside the air chamber 8, i.e. by evacuating air from the chamber 8, the two membranes 5, 6 are deformed since the external pressure is greater than the pressure inside the air chamber 8 and pushes on the external surfaces of the membranes 5, 6. Therefore, the two membranes 5, 6 are going to move closer and the membrane distance d is reduced. In particular, in a second configuration (b), air is evacuated (or totally evacuated) from the air chamber 8 such that the air chamber 8 is at a much lower pressure compared to the pressure exerted on the external surfaces of the membranes 5, 6. The air evacuation is indicated by the arrow in the vacuum port 10 pointing to the left. Accordingly, the atmospheric pressure deforms the two membranes 5, 6 so that they press or support each other, i.e. the membranes 5, 6 touch each other, and the membrane distance d is zero. In particular, the first deformable membrane 5 and the second deformable membrane 6 are deformed and stretched towards the central portion 9 of the walls 3 of the housing 2. Here, the first deformable membrane 5 is, at least partially, in contact with the second deformable membrane 6 to form a stretching region. The two opposite horizontal small arrows at the membranes 5, 6 indicate the stretching action due to the vacuumed air chamber 8, whereas the two opposite vertical small arrows at the membranes 5, 6 indicate the external pressure exerted on the membrane surfaces.

In examples, the first deformable membrane 5 or the second deformable membrane 6 is a deformable cell substrate 7 for growing cultured cells and a portion of the hollow region 4 represents a cell seeding chamber. With reference to FIG. 1, the first deformable membrane 5 (or upper membrane) is used as a deformable cell substrate 7, meaning that cells to be analyzed are seeded and growth on the external surface of the first membrane 5. Accordingly, the hollow region 4 defined by the first deformable membrane 5 and the lateral walls 3 of the housing 2 represents a cell seeding chamber.

The air chamber 8 has an average length I and an average height h. For example, the air chamber 8 can have a cylindrical shape so that the average length I represents the diameter of the cylinder base and the average height h represents the height of said cylinder.

In examples, the height h of the air chamber 8 is the predefined membrane distance d between the first deformable membrane 5 and the second deformable membrane 6 in the first configuration. As a matter of fact, if the pressure inside the air chamber 8 is equal or almost equal to the external pressure, the membranes 5, 6 are flat and their distance corresponds to the height of the air chamber 8, for example the cylinder height.

The ratio between the height h and the length l of the air chamber 8 can assume any reasonable value. However, in examples, the ratio between the height h and the length l of the air chamber 8 is comprised between 4:40 (i.e. 1/10) and 8:40 (i.e. ⅕), in particular 6:40 (i.e. 3/20).

In examples, the maximum stretching level of the cell stretching device 1 increases as a function of at least one of the length l of the air chamber 8, the membrane distance d, and the membrane thickness, at a fixed vacuum value inside the air chamber 8. The maximum stretching level is defined as the maximum possible stretching effect achieved on the membrane as a consequence of maximum pressure reduction inside the air chamber 8.

As a matter of fact, the maximum stretching level increases by increasing the value of the membrane distance d, the membrane thickness being the same. The maximum stretching level increases by reducing the membrane thickness, the value of the membrane distance d being the same.

In examples, the thickness of the first deformable membrane 5 and/or of the second deformable membrane 6 is comprised between 0.1 μm and 2000 μm, preferably between 1 μm and 1000 μm, in particular 100 μm or 200 μm. Specifically, it is noted that an optimal maximum stretching level is achieved with a membrane thickness of 100 μm or close to 100 μm.

In examples, the predefined membrane distance d in the first configuration is comprised between 0.1 mm and 200 mm, preferably 1 mm to 100 mm in particular 4 mm or 6 mm. Specifically, it is noted that an optimal maximum stretching level is achieved with a membrane distance d of 4 mm or close to 4 mm.

In examples, the first deformable membrane 5 and/or the second deformable membrane 6 has a diameter comprised between 1 mm and 100 mm, in particular 40 mm or 2 mm, 4 mm, 8 mm, 16 mm, 35 mm.

In examples, the housing 2 comprises a protruded region 11 for receiving a portion of the first deformable membrane 5 and of the second deformable membrane 6 when stretched in the second configuration. As shown in FIG. 1, the protruded region 11 is defined by the central portion 9 of the walls 3 of the housing 2 that confine the air chamber 8. In particular, the protruded region 11 represents an enlarged part or a recess of the housing 2 to house a part of the deformable membranes 5, 6 when stretched. For example, if the housing 2 has a cylindrical shape, the air chamber 8 and the hollow region/cell seeding chamber 4 have both a cylindrical hollow structure, whereas the cylindrical structure defining the air chamber 8 has a larger base compared to the cylindrical structure defining the hollow region/cell seeding chamber 4. The difference in dimension of the base between these two cylindrical structures represents the extension of the protruded region 11.

In examples, the stretching region is a flat region and in the second configuration the first and the second deformable membranes 5, 6 are exclusively in contact with each other and with the walls 3 of the housing 2. In other words, the flatness of the stretching region and therefore of the cell substrate 7 facilitates the adhesion of the cells on the membrane 5, 6. Also, the particular configuration, wherein the two membranes are only in contact with each other (i.e. one membrane support the other membrane), eliminates the necessity of a loading post.

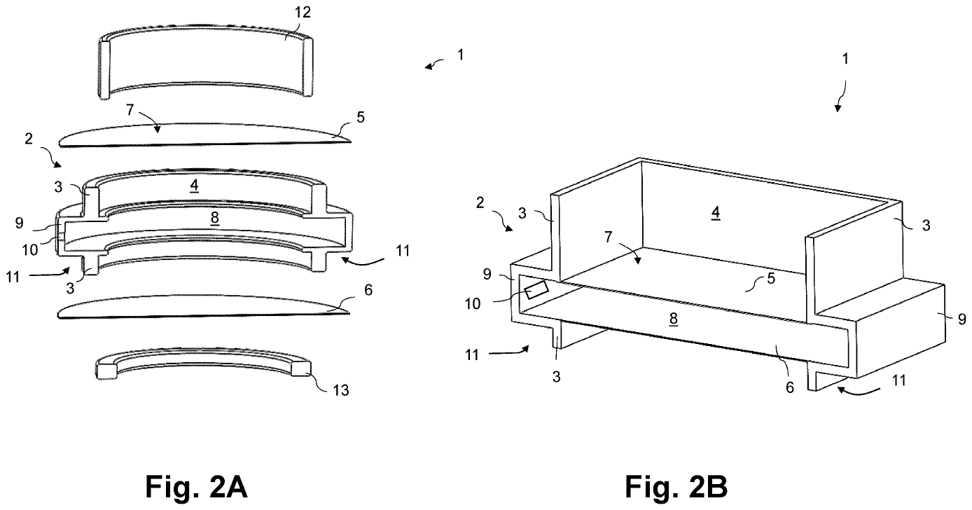

### FIG. 2a shows an exploded view (in cross section) of the cell stretching device 1 according to an example.

In example, the transversal cross-section of the housing 2 is circular. In particular, the housing 2 has a cylindrical structure comprising a cylindrical air chamber 8 and a cylindrical cell seeding chamber 4. The two chambers are coaxial and the air chamber 8 is larger than the cell seeding chamber 4. In particular, the difference in size between these two chambers is represented by the protruding region 11 as explained above. The circular air chamber 8 can be connected to a vacuum pump through the vacuum port 10 located at one side of the central portion 9 of walls 3 of the housing 2. Since the protruding region 11 extends all around the central portion 9 of the housing 2, this device is suitable for a biaxial stretching.

In examples, the device 1 comprises upper fixing means 12 for fixing the first deformable membrane 5 to the housing 2 and lower fixing means 13 for fixing the second deformable membrane 6 to the housing 2. The fixing means 12, 13 are configured to fix the membranes 5, 6 to the housing 2 in a removable way. In one example, the upper fixing means 12 and the lower fixing means 13 can be circular rings configured to clamp the first membrane 5 and the second membrane 6 to the housing, respectively. For this purpose the diameter of the circular rings can be lower of the diameter of the housing 2. In particular, the upper fixing means 12 is configured to clamp the first deformable membrane 5 to an upper part of the housing 2, for example at the cell seeding chamber 4. The lower fixing means 13 is configured to clamp the second deformable membrane 6 to a lower part of the housing 2, for example opposite to the cell seeding chamber 4. It is noted that the fixing means can be of any nature and can have any shape and dimension provided that they are suitable to fix the membranes 5, 6 to the housing 2, preferably in a removable way. For example, the fixing means can comprise gripping tools, adhesive tools, snap-fit tools, etc.

FIG. 2B shows a perspective view of the cell stretching device 1 according to another example. In this example, the transversal cross-section of the housing 2 is not circular but rather rectangular. In particular, the housing 2 has a polyhedric structure comprising a rectangular parallelepiped air chamber 8 and a rectangular parallelepiped cell seeding chamber 4. In addition, differently from the device of FIG. 2A, the protruding region 11 does not extend all around the central portion 9 of the housing 2, but extends only along two opposing sides of the central portion 9. Accordingly, the device of FIG. 2B is suitable for an uniaxial stretching. It is noted that, with the exception of the shape of the housing 2, the device of FIG. 2B comprises all the technical characteristics of the device of FIG. 2A. Therefore, all the characteristics defined in the following with reference to the configuration of the device 1 of FIG. 2A also apply to the configuration of the device 1 of FIG. 2B.

In examples, the first deformable membrane 5 and/or the second deformable membrane 6 is fixed to the housing 2 to form a sealing region between the housing 2 and said first and/or second deformable membrane 5, 6. In this way, the air chamber 8 is air sealed from outside. For example, with reference to FIG. 2, the sealing region is advantageously a circular region at the contact area between the housing 2, i.e. the walls 3, and the fixing means 12, 13.

In examples, the the housing 2 is made of plastic. In particular, the housing 2 can be a one-piece element made of a polymeric material or other material suitable to be printed by means of a 3D-printer. This implies that the device 1 can be mass produced easily by other industrial process and using different materials. The housing can also be made of different materials which are not necessary apt to be printed with a 3D-printer but are suitable for the purpose of the present invention. The fixing means 12, 13 can advantageously be made of the same material of the housing 2.

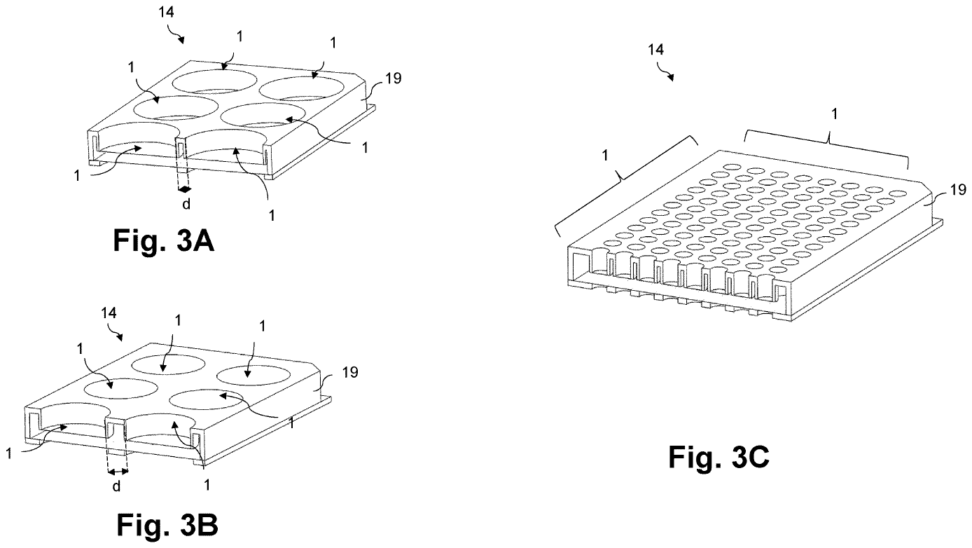

FIGS. 3A, 3B and 3C show two plates 14 for cell stretching according to two different examples. In particular, the plate 14 of FIGS. 3A and 3B comprises a plurality of cell stretching devices 1 arranged in rows and columns. Specifically, a frame is configured to position six devices 1 in an arrangement 2×3. On the other hand, the plate 14 of FIG. 3C comprises a frame configured to position ninety-six devices 1 in an arrangement 8×12. The first row of each plate 14 is cut to show that the present cell stretching device 1 and in particular the hollow region/cell seeding chamber 4 can be used as a well for hosting a culture cell substrate, possibly immersed in a growth medium.

The plates 14 of FIGS. 3A and 3B are basically the same. The only difference consists in the distance d between two consecutive devices 1. As shown in the figures, the distance d of the plate 14 of FIG. 3B is larger than the distance d of the plate 14 of FIG. 3A. By increasing the value of distance d, the stretching level is increased.

These plates 14 represent standard 6-wells and 96-wells plates used in laboratory for cell analysis. It is clear that these are just two examples and that other arrangements are possible, such as for example a 24-wells plate. In an example, the vacuum chamber of the plurality of cell stretching devices 1 of the plate 14 are connected to each other so that a plate vacuum port (not shown in the figure) is present, e.g. at a side of the frame 19 of the plate 14. Accordingly, the plate 14 can be connected to a vacuum system through said plate vacuum port. The connection from each chamber to the plate vacuum port can be carried out by a vacuum duct system inside the frame 19. Advantageously, the chambers are connected to a single plate vacuum port. However, a plurality of plate vacuum ports can be present, each plate vacuum port of said plurality being connected to a row or a column of cell stretching devices 1 (i.e. of vacuum ports 10) as arranged in the plate 14.

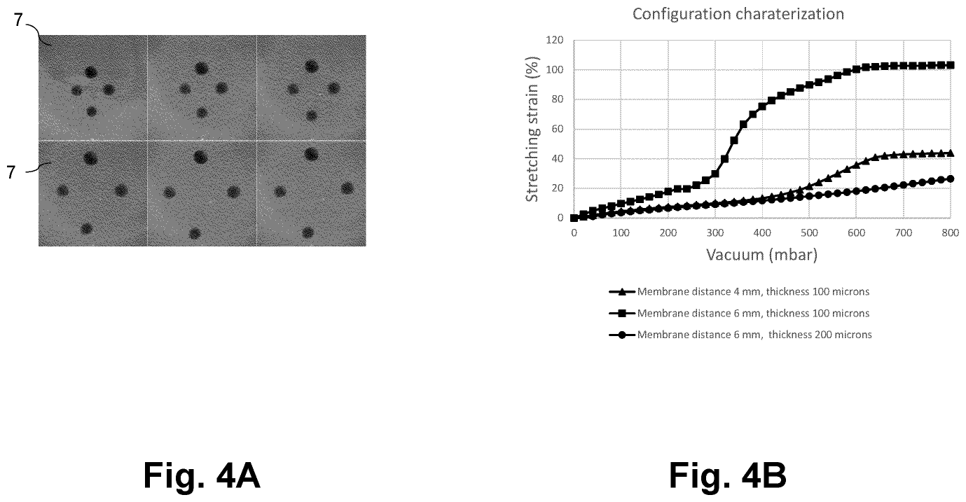

FIGS. 4A-B illustrate a characterization of configuration of membrane distance d and membrane thickness (membrane size is fixed at 40 mm in diameter) at different vacuum pressure values. FIG. 4A shows the cell substrate 7 (marked by four black ink dots) been stretched as vacuum increase from 0 to 800 mbar (membrane distance 6 mm and thickness 100 μm). FIG. 4B shows the stretching level (strain) (y-axis) of different configurations versus different vacuum pressure values (x-axis). It is noted from this figure that the stretching level at a fixed vacuum depends on the thickness, size, and distance of the two membranes 5, 6. In one example, with a membrane thickness of 100 μm thickness, a membrane diameter of 40 mm and membrane distance d of 6 mm, the maximum stretching level can increase to 100% after 600 mbar vacuum.

As shown in FIG. 4B, the maximum stretching level of the cell stretching device is determined by the size of the air chamber 8 and the thickness of the membrane 5, 6. Basically, by increasing the size of the air chamber 8, the maximum stretching level increases. As described above, in one configuration, the stretching level can increase to 100%, meaning that the membrane's surface is doubled by deformation Accordingly, the configuration of the device 1 can be scaled up to stretch large cell populations for biochemical and genetic analysis. The device 1 can also be miniaturized and used in assays for drug screening.

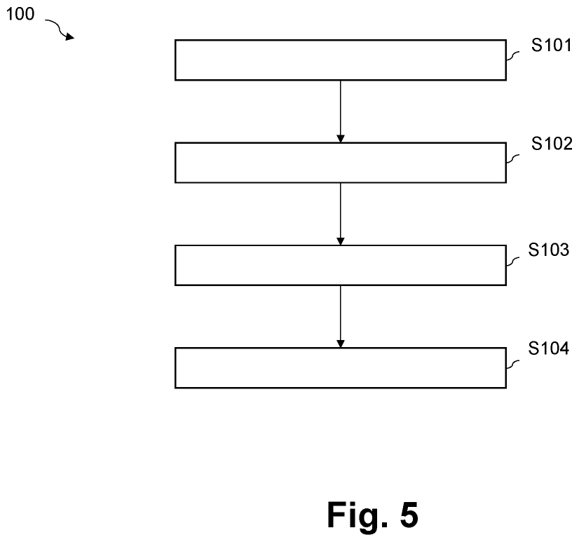

FIG. 5 schematically shows the steps of the method 100 for cell stretching using the device 1 or the plate 14 described above.

At step S101, at least one cell stretching device 1 or the plate 14 for cell stretching is provided. Then, one of the first or the second deformable membrane 5, 6 are seeded with cells to be analyzed, thereby forming a deformable cell substrate 7 (at step S102). At step S103, the cells are incubated for a first predetermined period of time and at step S104 the cell substrate 7 is stretched by cyclic aspiration and release of air through the vacuum port 10 of the device 1 or plate 14 for a second period of time.

In examples, the cyclic aspiration and release of air occurs by a vacuum pressure variation inside the air chamber 8 of the device 1 or plate 14, wherein in particular the vacuum pressure varies between 0 mbar and 800 mbar.

The device 1 is designed to enclose two flexible membranes 5, 6 (thickness 0.005 inches, commercialized by SMI Specialty Manufacturing Inc.) and the membrane stretching is achieved by cyclic aspiration and release of air through a negative and a positive air pump. Following device assembly and sterilization (by rinsing it with 70% EtOH), the upper membrane is coated with fibronectin (10 μg/ml), incubated at 37 °C for 1 hour and washed three times with 1x PBS sterile. IMR 90 fibroblasts are seeded in the cell chamber 4 (200.000/device) and incubated overnight before starting the stretching. The devices 1 are controlled by a MAT pipeline (Fluigent) programmed to exert a 20% cyclic stretch at 0.2 Hz frequency for a total of 6 hours inside in a humidified atmosphere (5% CO2, 37 °C). Cells seeded on non-stretched devices are used as control. Following 6 h of continuous cyclic cell stretching, cells are washed once with 1X PBS, then lysed by adding 150 μl of 2X BOLTTMLDS buffer complemented with 100 mM DTT and scraping the membrane surface with a cell scraper. Lysates are collected in 1.5 ml tubes on ice, sonicated for 10 s at 4 °C then boiled for 10 minutes at 95 °C. Samples are stored at −20 °C until ready to be analysed by western blot.

According to the results, quantifications of H3K9me3 and H3K27me3 relative to total H3 show increased chromatin condensation in cells treated with ATMi and exposed to cyclic stretching.

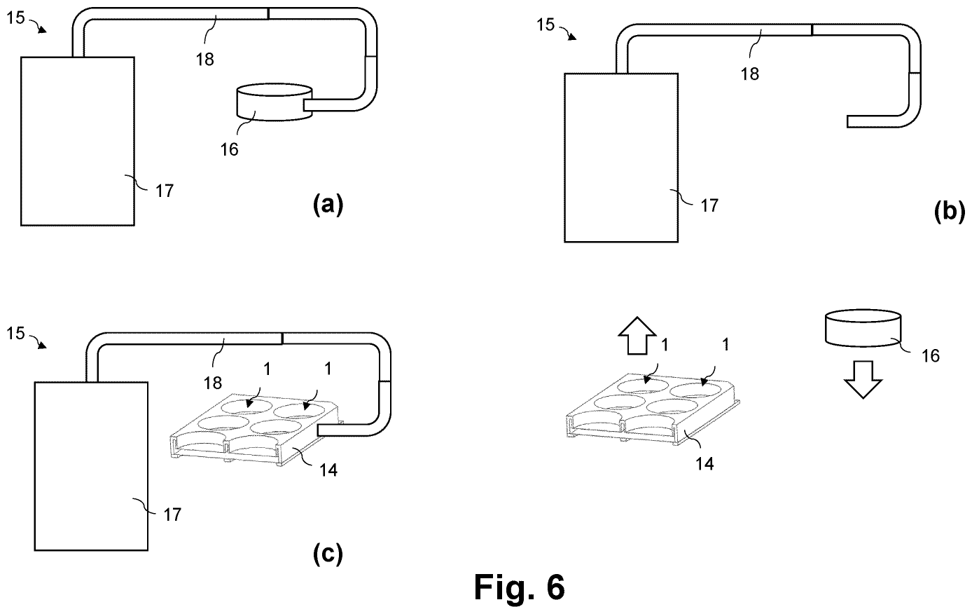

FIG. 6 illustrates the steps of a process for retrofitting a standard cell stretching system. In particular, this process is suitable for a cell stretching system that uses a vacuum unit 15 for generating a stretching on a deformable cell substrate. The cell stretching system comprises an apparatus 16 supporting the deformable cell substrate and being configured to be connected to said vacuum unit 15. As shown in FIG. 6(a), the vacuum unit 15 comprises at least a pump unit 17 and a piping system 18 connecting the pump unit 17 to the apparatus 16. The process comprises replacing the apparatus 16 with the cell stretching device 1 or the cell stretching plate 14 previously described. The replacement occurs by simply connecting the vacuum unit 15 to the vacuum port 10 of the cell stretching device 1 (FIG. 6 (b)-(c)). In other words, the performance of a standard cell stretching system can be improved by using the cell stretching device 1 or the cell stretching plate 14 here described, without the necessity of replacing or adding the vacuum unit 15, thereby saving overall costs.

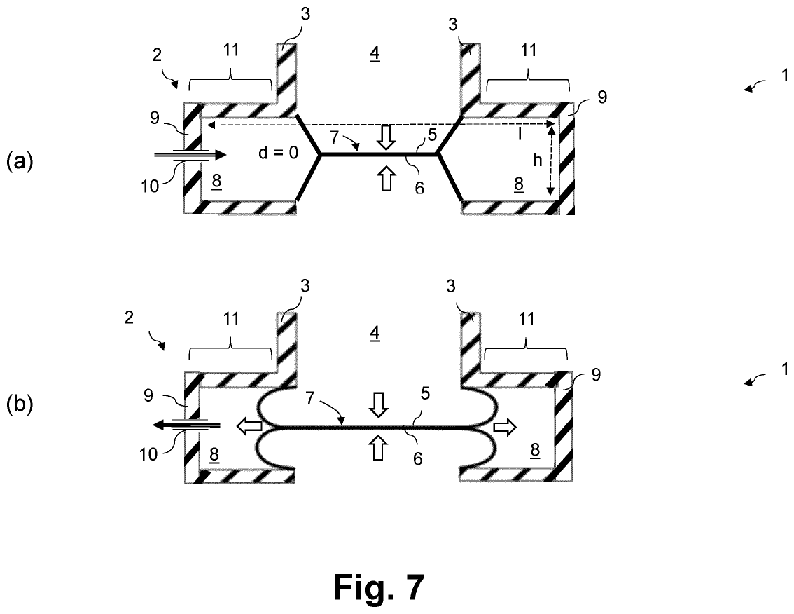

With reference to FIG. 7, an alternative cell stretching device 1 is shown. It is noted that the device 1 of this figure is similar to that of FIG. 1 and basically functions in the same way. The only difference consists in the arrangement of the first and second membrane 5, 6 in the first configuration (a) when air is not evacuated (or partially evacuated) from the air chamber 8. In particular, the two membranes are fused together in the first configuration (a), i.e., before stretching. Besides this difference related to the membranes configuration, the other components and characteristics of the device of FIG. 1 also apply to the device of FIG. 7. For example, similar to the device 1 of FIG. 1, the cell stretching device 1 of FIG. 7 comprises a housing 2 having basically a cylindrical shape defined by confining walls 3. The housing 2 has a hollow structure and comprises a hollow region 4. The device 1 comprises two deformable membranes, a first membrane 5 and a second membrane 6 both fixed to the walls 3 of the housing 2. The first membrane 5 and the second membrane 6 define, together with a central portion 9 of the walls 3 of the housing 2, an air chamber 8. In other words, the air chamber 8 is formed between the two membranes 5, 6 and the central portion 9 of the walls 3 of the housing 2 in an internal region of the housing 2. In order to evacuate air from the air chamber 8 and therefore to change, in particular reduce, the air pressure inside the chamber 8, the central portion 9 comprises at least a vacuum port 10. Accordingly, the air chamber 8 can be connected to a vacuum unit comprising a vacuum pump through the vacuum port 10. As the air chamber 10 is vacuumed, the membranes 5, 6 are deformed by atmospheric pressure and pulled laterally to form a flat stretched region.

The device of FIG. 7 basically behaves like the device of FIG. 1. In a first configuration (a), the air pressure inside the air chamber 8 is such that the two membranes 5, 6 are not deformed. For example, air is not evacuated (or partially evacuated) from the air chamber 8, meaning that the air chamber 8 is at the atmospheric pressure or at a pressure value close to the atmospheric pressure such that the pressure exerted on the internal surfaces of the two membranes 5, 6 compensates (or almost compensate) the pressure exerted on the external surfaces of the two membranes 5, 6, as schematically shown by the vertical opposing arrows in the figure. The internal surface of the membranes 5, 6 indicates the side of the membranes 5, 6 facing the air chamber 8, whereas the external surface of the membranes 5, 6 indicates the side of the membranes 5, 6 opposite to the internal surface, i.e. outside the air chamber 8. The arrow shown in the vacuum port 10 pointing to the right in the figure means that in the first configuration air can be inserted into the air chamber 8 from outside in order to compensate the atmospheric pressure exerted on the external surfaces of the first and second membranes 5, 6, for example if the air was first evacuated from the chamber 8. In the first configuration (a), differently from the device of FIG. 1, the first deformable membrane 5 is attached to the second deformable membrane 6. The two membranes form a common flat surface. In other words, the distance d between the membranes is zero (d=0). If the pressure inside the air chamber 8 equals the external pressure on the membranes 5, 6, e.g., the atmospheric pressure, the two membranes 5, 6 remain in the first configuration. By reducing the air pressure inside the air chamber 8, i.e. by evacuating air from the chamber 8, the two membranes 5, 6 are deformed since the external pressure is greater than the pressure inside the air chamber 8 and pushes on the external surfaces of the membranes 5, 6. In particular, in a second configuration (b), air is evacuated (or totally evacuated) from the air chamber 8 such that the air chamber 8 is at a much lower pressure compared to the pressure exerted on the external surfaces of the membranes 5, 6. The air evacuation is indicated by the arrow in the vacuum port 10 pointing to the left. In particular, the first deformable membrane 5 and the second deformable membrane 6 are deformed and stretched towards the central portion 9 of the walls 3 of the housing 2. The two opposite horizontal small arrows at the membranes 5, 6 indicate the stretching action due to the vacuumed air chamber 8, whereas the two opposite vertical small arrows at the membranes 5, 6 indicate the external pressure exerted on the membrane surfaces.

Accordingly, in an alternative embodiment, a cell stretching device 1 can be provided comprising:
a housing 2 having walls 3 defining a hollow region 4;
a first deformable membrane 5 and a second deformable membrane 6, both the first deformable membrane 5 and the second deformable membrane 6 being located in the housing 2 and being fixable to the walls 3 of the housing 2, wherein the first membrane 5 is in contact (i.e. fused) at least partially with the second membrane 6; and
an air chamber 8 formed in an internal region of the housing 2 and defined by the first deformable membrane 5, the second deformable membrane 6, and a central portion 9 of the walls 3 of the housing 2, said central portion 9 of the walls 3 comprising at least a vacuum port 10 for evacuating air from the air chamber 8 and to determine a pressure variation inside the air chamber 8,
wherein in a first configuration the air pressure inside the air chamber 8 is such that the first deformable membrane 5 in contact with the second deformable membrane 6 is not deformed, and in a second configuration the air pressure inside the air chamber 8 is reduced such that the first deformable membrane 5 and the second deformable membrane 6 are deformed and stretched towards the central portion 9 of the walls 3 of the housing 2 to form a stretching region.

All the characteristics and applications described in FIGS. 1 to 6 also apply to this alternative embodiment of the cell stretching device 1.

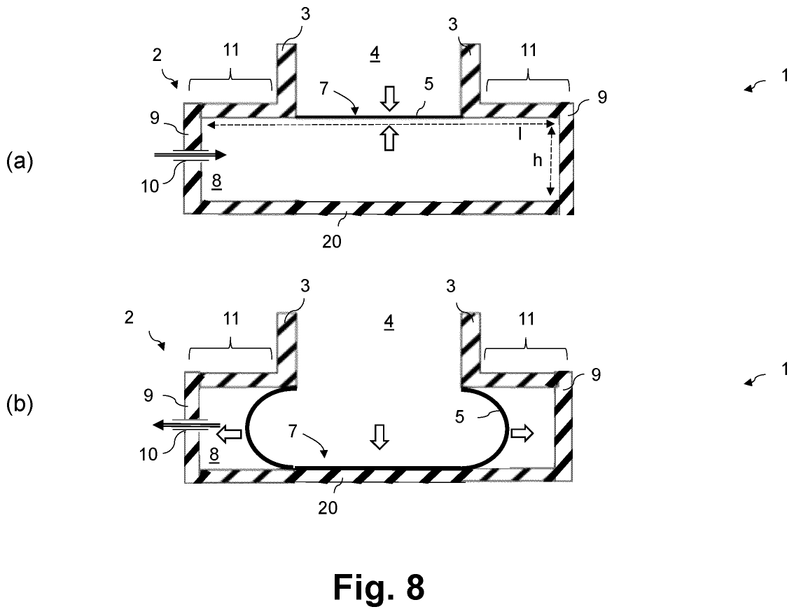

With reference to FIG. 8, a further alternative cell stretching device 1 is shown. It is noted that the device 1 of this figure is similar to that of FIGS. 1 and 7 and basically functions in the same way. The device 1 of FIG. 8 differs form the device 1 of FIG. 1 in that only one deformable membrane 5 is present. The second membrane 6 of the device of FIG. 1 is replaced by a portion of the housing 2, i.e. the base 20 of the housing. In the first configuration (a) air is not evacuated (or partially evacuated) from the air chamber 8. In particular, the single membrane 5 is flat. Besides this difference related to the membranes configuration, the other components and characteristics of the device of FIG. 1 also apply to the device of FIG. 8. For example, similar to the device 1 of FIG. 1, the cell stretching device 1 of FIG. 8 comprises a housing 2 having basically a cylindrical shape defined by confining walls 3. The housing 2 has a hollow structure and comprises a hollow region 4. The device 1 comprises a deformable membrane 5 fixed to the walls 3 of the housing 2. The membrane 5 faces the base 20 of the housing 2 and defines, together with said base 20 and a central portion 9 of the walls 3 of the housing 2, an air chamber 8. In other words, the air chamber 8 is formed between the membrane 5, the base 20 and the central portion 9 of the walls 3 of the housing 2 in an internal region of the housing 2. In order to evacuate air from the air chamber 8 and therefore to change, in particular reduce, the air pressure inside the chamber 8, the central portion 9 comprises at least a vacuum port 10. Accordingly, the air chamber 8 can be connected to a vacuum unit comprising a vacuum pump through the vacuum port 10. As the air chamber 10 is vacuumed, the membrane 5 is deformed by atmospheric pressure and is laterally pulled to form a flat and stretched region.

The device of FIG. 8 basically behaves like the device of FIG. 1. In a first configuration (a), the air pressure inside the air chamber 8 is such that the membrane 5 is not deformed. For example, air is not evacuated (or partially evacuated) from the air chamber 8, meaning that the air chamber 8 is at the atmospheric pressure or at a pressure value close to the atmospheric pressure such that the pressure exerted on the internal surface of the membrane 5 compensates (or almost compensate) the pressure exerted on the external surface of the membrane 5, as schematically shown by the vertical opposing arrows in the figure. The internal surface of the membrane 5 indicates the side of the membrane 5 facing the air chamber 8, whereas the external surface of the membrane 5 indicates the side of the membrane 5 opposite to the internal surface, i.e. outside the air chamber 8. The arrow shown in the vacuum port 10 pointing to the right in the figure means that in the first configuration air can be inserted into the air chamber 8 from outside in order to compensate the atmospheric pressure exerted on the external surfaces of the membrane 5, for example if the air was first evacuated from the chamber 8. In the first configuration (a), the membrane 5 is flat. By reducing the air pressure inside the air chamber 8, i.e. by evacuating air from the chamber 8, the membrane 5 is deformed since the external pressure is greater than the pressure inside the air chamber 8 and pushes on the external surfaces of the membrane 5. In particular, in a second configuration (b), air is evacuated (or totally evacuated) from the air chamber 8 such that the air chamber 8 is at a much lower pressure compared to the pressure exerted on the external surfaces of the membrane 5. The air evacuation is indicated by the arrow in the vacuum port 10 pointing to the left. In particular, the deformable membrane 5 is pushed towards the base 20 of the housing 2 and is stretched towards the central portion 9 of the walls 3 of the housing 2. The two opposite horizontal small arrows at the membrane 5 indicate the stretching action due to the vacuumed air chamber 8, whereas the vertical small arrow at the membranes 5 (in opposition to the reaction force of the base 20) indicate the external pressure exerted on the membrane surfaces. In this case, the use of a lubricant can be necessary.

Accordingly, in a further alternative a cell stretching device 1 can be provided comprising:
a housing 2 having walls 3 defining a hollow region 4;
a single deformable membrane 5 located in the housing 2 fixable to the walls 3 of the housing 2, wherein the membrane 5 faces a base 20 of the housing 2; and
an air chamber 8 formed in an internal region of the housing 2 and defined by the deformable membrane 5, the base 20 of the housing 2, and a central portion 9 of the walls 3 of the housing 2, said central portion 9 of the walls 3 comprising at least a vacuum port 10 for evacuating air from the air chamber 8 and to determine a pressure variation inside the air chamber 8,
wherein in a first configuration the air pressure inside the air chamber 8 is such that the deformable membrane 5 is flat and not deformed, and in a second configuration the air pressure inside the air chamber 8 is reduced such that the deformable membrane 5 is deformed to touch at least partially the base 20 of the housing 2 and it is stretched towards the central portion 9 of the walls 3 of the housing 2 to form a stretching region. All the characteristics and applications described in FIGS. 1 to 6 also apply to this further alternative embodiment of the cell stretching device 1.

It is noted that the two embodiments described in FIGS. 7 and 8 are two alternative embodiments of the device 1 described in FIG. 1 and functions according to the same stretching principle. Accordingly, a plate 14 for cell stretching can be provided comprising a plurality of cell stretching devices 1 as shown in FIG. 1, and/or the stretching devices 1 as shown in FIG. 7 and/or the stretching devices 1 as shown in FIG. 8, and at least a frame 19 configured to position each of said plurality of cell stretching devices 1 arranged in rows and columns. Also, a method 100 for cell stretching using the device 1 as shown in FIG. 7 and/or the device 1 as shown in FIG. 8 can be provided. Similarly considerations can be done for a process for retrofitting a cell stretching system as described above.

Although a variety of techniques and examples of such techniques have been described herein, these are provided by way of example only and many variations and modifications on such examples will be apparent to the skilled person and fall within the spirit and scope of the present invention, which is defined by the appended claims and their equivalents.

1. A cell stretching device comprising:
a housing having walls defining a hollow region;
a first deformable membrane and a second deformable membrane facing the first deformable membrane both the first deformable membrane and the second deformable membrane being located in the housing and being fixable to the walls of the housing and
an air chamber formed in an internal region of the housing and defined by the first deformable membrane the second deformable membrane and a central portion of the walls of the housing said central portion of the walls comprising a vacuum port for evacuating air from the air chamber and to determine a pressure variation inside the air chamber,
wherein in a first configuration, the air pressure inside the air chamber is such that the first deformable membrane is separated from the second deformable membrane at a predefined membrane distance (d), and in a second configuration the air pressure inside the air chamber is reduced such that the first deformable membrane and the second deformable membrane are deformed and stretched towards the central portion of the walls of the housing and such that the first deformable membrane is, at least partially, in contact with the second deformable membrane to form a stretching region.

2. The device according to claim 1, wherein the first deformable membrane or the second deformable membrane is a deformable cell substrate for growing cultured cells and a central portion of the hollow region represents a cell seeding chamber.

3. The device according to claim 1, wherein the air chamber has an average length (l) and an average height (h) and wherein:
a. the height (h) of the air chamber is the predefined membrane distance (d) between the first deformable membrane and the second deformable membrane in the first configuration, and/or
b. the ratio between said height (h) and said length (l) is comprised between 4:40 and 8:40, in particular 6:40; and/or
c. the maximum stretching level of the cell stretching device increases as a function of at least one of said length (l), the membrane distance (d), and the membrane thickness, at a fixed vacuum value inside the air chamber.

4. The device according to claim 1, wherein the thickness of the first deformable membrane and/or of the second deformable membrane is comprised between 0.1 μm and 1000 μm.

5. The device according to clam 1, wherein the predefined membrane distance (d) in the first configuration is between 0.1 mm and 100 mm.

6. The device according to clam 1, wherein the first deformable membrane and/or the second deformable membrane is fixed to the housing to form a sealing region between the housing and said first and/or second deformable membrane.

7. The device according to clam 1, further comprising upper fixing means for fixing the first deformable membrane to the housing and lower fixing means for fixing the second deformable membrane to the housing.

8. The device according to claim 1, wherein the housing comprises a protruded region for receiving a portion of the first deformable membrane and of the second deformable membrane when stretched in the second configuration.

9. The device according to claim 1, wherein
a. the transversal cross-section of the housing is circular for a multiaxial stretching; or
b. the transversal cross-section of the housing is rectangular for an uniaxial stretching; and/or
c. the housing is made of plastic.

10. The device according to claim 1, wherein the stretching region is a flat region and wherein in the second configuration the first and the second deformable membranes are exclusively in contact with each other and with the walls of the housing.

11. The device according to claim 1, wherein the first deformable membrane and/or the second deformable membrane has a diameter of between 1 mm and 100 mm, 16 mm, 35 mm.

12. A plate for cell stretching comprising:
a plurality of cell stretching devices according to claim 1, and frame configured to position each of the plurality of cell stretching devices arranged in rows and columns.

13. A method for cell stretching comprising:
providing a cell stretching device of claim 1 for cell stretching;
seeding one of the first or the second deformable membrane with cells to be analyzed, thereby forming a deformable cell substrate
incubating the cells for a first period of time; and
stretching the cell substrate by cyclic aspiration and release of air through the vacuum port of the device for a second period of time.

14. The method of claim 13, wherein the cyclic aspiration and release of air occurs by a vacuum pressure variation inside the air chamber of the device.

15. A process for retrofitting a cell stretching system that uses a vacuum unit for generating a stretching on a deformable cell substrate, said cell stretching system comprising an apparatus supporting the deformable cell substrate and being configured to be connected to said vacuum unit the process comprising replacing the apparatus with the cell stretching device according to claim 1 by connecting the vacuum unit to the vacuum port of the cell stretching device.

16. The device of claim 4, wherein the thickness of the first deformable membrane and/or of the second deformable membrane is 100 μm or 200 μm.

17. The device of clam 5, wherein the predefined membrane distance (d) in the first configuration is 4 mm or 6 mm.

18. The device of claim 11, wherein the first deformable membrane and/or the second deformable membrane has a diameter selected from the group consisting of 40 mm, 2 mm, 4 mm, 8 mm, 16 mm, 35 mm.

19. The method of claim 14, wherein the vacuum pressure varies between 0 mbar and 800 mbar.
